(19) United States
(12) Plant Patent Application Publication
(10) Pub. No.: US 2008/0184433 P1
(43) Pub. Date: Jul. 31, 2008

(54) Verbena plant named 'Pechena'

(75) Inventor: Henricus G.W. Stemkens, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/640,593

(22) Filed: Dec. 18, 2006

(51) Int. Cl. A01H 5/00

(52) U.S. Cl. PLT/308

(57) ABSTRACT

A new Verbena plant particularly distinguished by its salmon flower, early flowering, and a habit that is upright and later spreading.

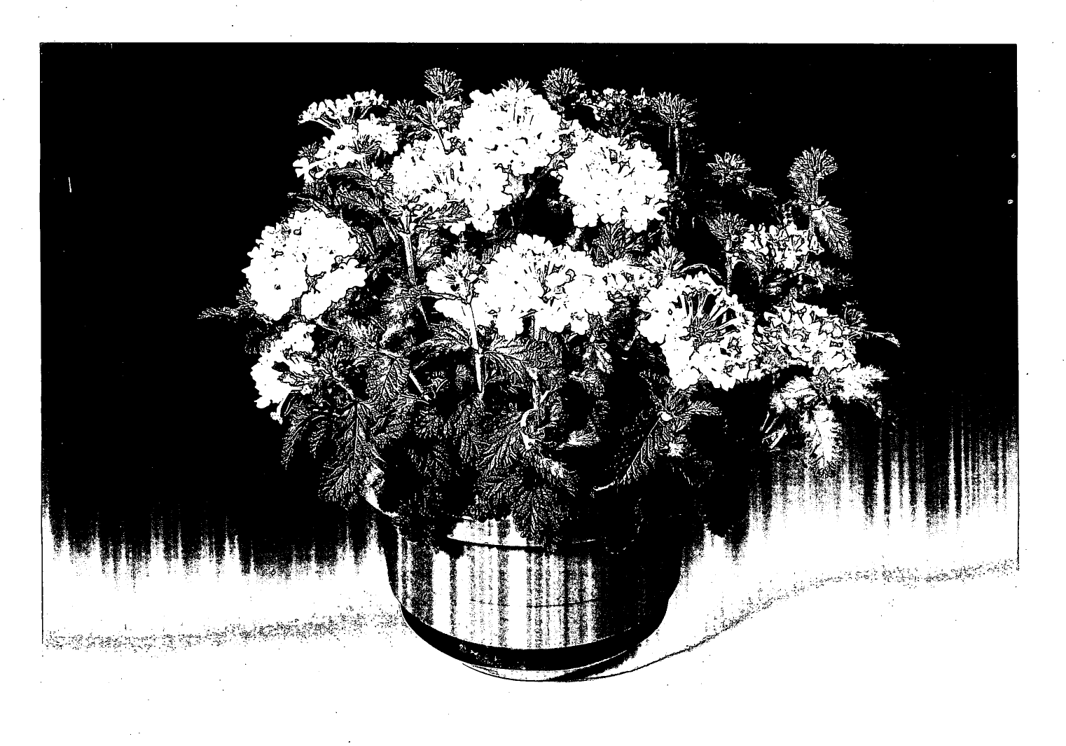

### LATIN NAME OF THE GENUS AND SPECIES OF THE PLANT CLAIMED

Verbena x hybrida

### VARIETAL DENOMINATION

‘Pechena’

### BACKGROUND OF THE NEW PLANT

The present invention comprises a new distinct cultivar of Verbena, botanically known as Verbena x hybrida.

The new cultivar is originated from the self-pollination of ‘D0806-1’ as female parent and as the male or pollen parent. ‘D0806-1’ is an apricot flowering Verbena. ‘D0806-1’ is not commercially available and is not known by any synonyms. ‘D0806-1’ has not been patented.

As a result of this self-pollination the present cultivar was created in 2002 in Enkhuizen, Netherlands and has been repeatedly asexually reproduced by cuttings in Enkhuizen, Netherlands and Sarrians, France over a two year period. The new variety is stable and reproduces true to type in successive generations of asexual reproduction.

This new Verbena plant is an annual in most climatical zones in the US, only in zones 9 and 10 it is a perennial plant.

### DESCRIPTION OF THE DRAWING

This new Verbena plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Verbena. The data that define these characteristics were collected from asexual reproductions carried out in Enkhuizen, Netherlands. The plant history was taken on 14 week old plants, blossomed under natural light in a greenhouse, and grown in a 10.5 cm container. Color readings were taken in the greenhouse under ambient light.

Color references are primarily to the R.H.S. Colour Chart of The Royal Horticultural Society of London.

TABLE 1
DIFFERENCES BETWEEN THE NEW CULTIVAR ‘PECHENA,’
ITS PARENT, AND THE MOST RESEMBLING VARIETY

| | ‘Pechena’ | ‘D0806-1’ | ‘Lanai Peach’ |
|---|---|---|---|
| Flower color | Salmon-red | Apricot | Salmon |
| Flower shape | Undulate | Flat | Flat |
| Earliness | Mid early | Early | Very late |

The commercial name of the most resembling variety is ‘Lanai Peach.’ This variety has protected name “Lan Peachy’ and is patented as U.S. Plant Pat. No. 15,536.

### THE PLANT

Classification — botanical: Verbena x hybrida
Parentage — female and male parent: A seedling named ‘D0806-1’ is one of our seedlings from our D-generation of plants.
   Growth habit.—Upright, decumbent.
   Plant height.—15-26 cm.
   Spreading area of plant.—40-50 cm.
   Branching character.—Freely branching and lateral branching at every node.
   Number of branches per plant.—28-34.
   Blooming period.—From March until November.
The stem:
   Diameter.—2 mm.
   Length.—14-24 cm.
   Shape.—Tetragonal.
   Anthocyanin pigmentation.—Absent.
   Color of the stem.—146A.
   Length of internode.—33 mm.
   Texture.—Smooth.
   Pubescence.—Pubescent.
   Length lateral branches.—35-45 cm.
The foliage:
   Phyllotaxis.—Opposite.
   Shape of blade.—Lanceolate.
   Texture.—Upper side: Smooth Lower side: Smooth.
   Venation.—Pinnate.
   Leaf margin.—Crenate.
   Leaf base.—Obtuse.
   Leaf apex.—Attenuate.
   Length.—18-34 mm.
   Width.—14-20 mm.
   Depth of incision.—3-5 mm.
   Number of incisions.—12-18 per leaf.
   Color.—Upper side: 141B Lower side: 141C.
   Pubescence.—Slightly pubescent.
   Length of petiole.—2 mm.
   Petiole surface structure.—Slightly pubescent.
   Petiole diameter.—1 mm.
   Petiole coloration.—141B, both surfaces.
The bud:
   Peduncle length.—22 to 45 mm.
   Peduncle diameter.—2.5 mm.
   Peduncle color.—141C.
   Bud size.—Diameter: 2 mm Length: 4-6 mm.
   Bud shape.—Elongated and ovate.
   Bud color.—37A.
   Sepals.—Color (upper side): 141B Color (lower side): 141 C Form: Upright Number: 5, fused Length: 6-8 mm Width: 2 mm Shape: Elongated Apex: Emarginate Base: Fused Margin: Entire.
The flower:
   Flower diameter.—14-18 mm.
   Flower height.—16-24 mm.
   Flower tube length.—14-22 mm.
   Flower throat diameter.—2 mm.
   Flower throat color.—156B.
   Inflorescence.—Corymb.
   Flower.—Form Single, salverform; sessile on terminal corymbs.
   Petal color on opening.— Upper side: From 40D in the centre to 41 C on the edge Lower side: 37A.
   Petal color when mature.— Upper side: From 29A in the center to 29C on the edge Lower side: 37A a bit lighter.
   Petal color just before drop.— Upper side: 27A a bit darker Lower side: 27C.
   Overlapping of petals.—Separate.
   No. of petals.—Gamopetalous, 5 lobed.
   Petal apex.—Obcordate.
   Petal base.—Fused.
   Petal margin.—Entire.
   Petal surface texture.—Smooth.
   Size of the petal.— Length: 6-8 mm Width: 4-6 mm.
   Inflorescence.—Length: 18-24 mm Diameter: 24-30 mm.
   Calyx length.—7-9 mm.
   Calyx diameter.—3-4 mm.
   Anthocyanin pigmentation of calyx limb.—Absent.
   Color of the calyx.—141B.
   No. of flowers per inflorescence.—20-35 .
   Fragrance.—No fragrance.
   Bloom time of one inflorescence.—New florets continue to open in one corymb over a period of 14 days.
   Lastingness of one flower.—2-4 days.

### Reproductive organs:

Androecium:
   Stamens quantity.—4 .
   Anther shape.—Ovoid.
   Anther length.—1 mm.
   Anther color.—144C.
   Pollen amount.—No pollen.
Gynoecium:
   Pistils quantity.—1.
   Pistil length.—1-1.5 cm.
   Stigma shape.—Bi-lobed.
   Stigma color.—144C.
   Style length.—1.9cm.
   Style color.—144C.
   Ovary color.—144C.
The fruit and the seed: No fruit development or seed set has been observed to date.
Roots:
   Type of roots.—Fibrous, color 155B. Roots start to grow on every part of the stem that contacts the soil, so not only at the nodes.
Physiological and ecological characteristics:
   Disease/pest resistance.—Plants of the new Verbena have been noted to be resistant to especially powdery mildew.
   Temperature tolerance.—Plants of the new Verbena have been observed to tolerate temperatures from 2-35 degrees C.

What is claimed is:

1. A new and distinct variety of Verbena plant, substantially as illustrated and described herein.
